(19) United States
(12) Patent Application Publication
(10) Pub. No.: US 2016/0367137 A1
(43) Pub. Date: Dec. 22, 2016

(54) METHOD AND SYSTEM FOR DETECTING CARDIOPULMONARY ABNORMALITY

(71) Applicant: Jon-Chao HONG, Taipei City (TW)

(72) Inventors: Jon-Chao HONG, Taipei City (TW); Kai-Hsiang YANG, Taipei City (TW); Chao-Hsin WU, Taipei City (TW)

(21) Appl. No.: 15/185,318

(22) Filed: Jun. 17, 2016

(30) Foreign Application Priority Data
Jun. 22, 2015 (TW) 104119986

(51) Int. Cl. A61B 5/00; A61B 7/04; G08B 21/04

(52) U.S. Cl. CPC A61B 5/0004; G08B 21/0461; A61B 5/746; A61B 7/045; A61B 5/7282

(57) ABSTRACT

A system for detecting cardiopulmonary abnormality includes a mobile device and a processing device. The mobile device is capable of acquiring current cardiopulmonary data associated with current cardiopulmonary sounds from a user. The processing device is located remotely of and communicatively associated with the mobile device, receives the current cardiopulmonary data from the mobile device, determines whether the current cardiopulmonary data is associated with abnormal cardiopulmonary sounds, and transmits in real-time a message indicating cardiopulmonary abnormality to the mobile device upon determining that the current cardiopulmonary data is associated with abnormal cardiopulmonary sounds.

### CROSS-REFERENCE TO RELATED APPLICATION

This application claims priority to Taiwanese Application No. 104119986 filed on Jun. 22, 2015.

### FIELD

The disclosure relates to a method and a system for detecting in real-time cardiopulmonary abnormality of a user.

### BACKGROUND

A conventional system for detecting cardiopulmonary abnormality is usually located in a medical institute and thus the patients suffering from diseases relating to heart conditions or asthma need to go to the medical institute regularly to track and examine their cardiopulmonary condition.

### SUMMARY

Therefore, an object of the disclosure is to provide a system and a method for detecting and/or reporting in real-time cardiopulmonary abnormality of a user.

Accordingly, a system for detecting cardiopulmonary abnormality includes a mobile device and a processing device. The mobile device includes a cardiopulmonary data acquiring module for acquiring current cardiopulmonary data associated with current cardiopulmonary sounds from a user. The processing device is located remotely of and communicatively associated with the mobile device, receives the current cardiopulmonary data from the mobile device, determines whether the current cardiopulmonary data is associated with abnormal cardiopulmonary sounds, and transmits in real-time an alert message indicating cardiopulmonary abnormality to the mobile device upon determining that the current cardiopulmonary data is associated with abnormal cardiopulmonary sounds.

According to another aspect of this disclosure, a system for reporting cardiopulmonary abnormality includes a first mobile device, a second mobile device and a processing device. The first mobile device includes a cardiopulmonary data acquiring module that is for acquiring current cardiopulmonary data associated with current cardiopulmonary sounds from a user. The second mobile device is communicatively associated with the first mobile device. The processing device is located remotely of and communicatively associated with the first and second mobile devices, receives the current cardiopulmonary data from the first mobile device, and determines whether the current cardiopulmonary data is associated with abnormal cardiopulmonary sounds. The processing device transmits in real-time an alert message indicating cardiopulmonary abnormality to the second mobile device upon determining that the current cardiopulmonary data from the first mobile device is associated with abnormal cardiopulmonary sounds.

According to still another aspect of this disclosure, a method for detecting cardiopulmonary abnormality is provided. The method is to be implemented by a processing device that is located remotely of and communicatively associated with a mobile device and includes the steps of:
a) acquiring, by the processing device, current cardiopulmonary data associated with cardiopulmonary sounds from a user as acquired by the mobile device;
b) determining, by the processing device, whether the current cardiopulmonary data is associated with abnormal cardiopulmonary sounds; and
c) transmitting in real-time, by the processing device, an alert message indicating cardiopulmonary abnormality to the mobile device when it is determined in step b) that the current cardiopulmonary data is associated with abnormal cardiopulmonary sounds.

### BRIEF DESCRIPTION OF THE DRAWINGS

Other features and advantages of the disclosure will become apparent in the following detailed description of the embodiments with reference to the accompanying drawings, of which:

FIG. 1 is a schematic block diagram illustrating a system including a mobile device for detecting cardiopulmonary abnormality according to an embodiment of the present disclosure;

FIG. 2 is a schematic block diagram of a system including a plurality of mobile devices according to another embodiment of the present disclosure; and

FIG. 3 is a flow chart of a method for detecting cardiopulmonary abnormality according to an embodiment of the present disclosure.

### DETAILED DESCRIPTION

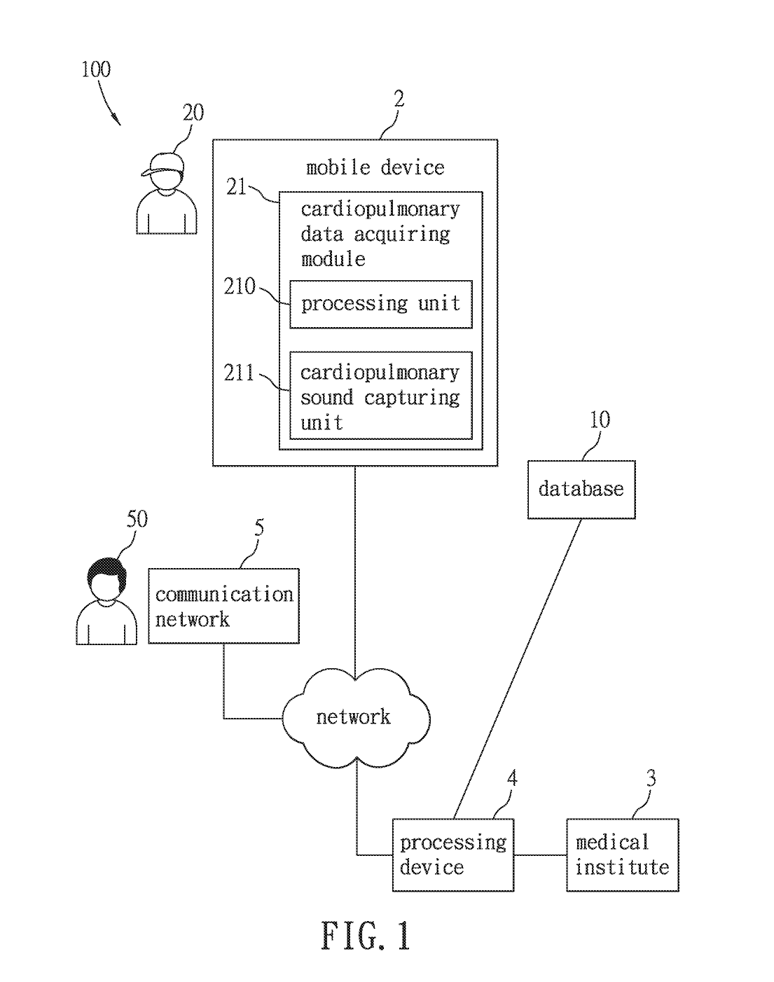

Referring to FIG. 1, a system 100 for detecting cardiopulmonary abnormality includes a database 10, a mobile device 2 and a processing device 4. The mobile device 2 includes a cardiopulmonary data acquiring module 21 that includes a processing unit 210 and a cardiopulmonary sound capturing unit 211.

The cardiopulmonary sound capturing unit 211 is for capturing current cardiopulmonary sounds from a user 20. The cardiopulmonary sound capturing unit 211 may be disposed on the user 20 at a position adjacent to a thoracic cavity, an abdominal cavity and a spinal cavity of the user 20 as desired. It can be appreciated that the cardiopulmonary sound capturing unit 211 can be implemented by a built-in microphone of the mobile device 2, an external microphone communicating with the mobile device 2 (e.g., via a direct connection therebetween), or other audio devices capable of capturing the current cardiopulmonary sounds from the user 20.

The processing unit 210 of the mobile device 2 is for converting the current cardiopulmonary sounds captured by the cardiopulmonary sound capturing unit 211 into current cardiopulmonary data. In this embodiment, the current cardiopulmonary data is represented by waveform and frequency.

The database 10 is accessible by the processing device 4 and stores a plurality of normal reference data associated with different normal cardiopulmonary sounds and a plurality of abnormal reference data associated with different abnormal cardiopulmonary sounds. Specifically, the normal reference sounds are captured from healthy persons and the abnormal reference sounds are captured from patient suffering from diseases/conditions relating to myocardial infarction, mitral valve prolapsed, asthma, etc.

The processing device 4 is located remotely of and communicatively associated with the mobile device 2, receives the current cardiopulmonary data from the mobile device 2, and determines whether the current cardiopulmonary data is associated with abnormal cardiopulmonary sounds through machine learning. For instance, the processing device 4 may be connected to the mobile device 2 over a network, such as the Internet, thereby being able to communicate therewith. In one embodiment, the processing device 4 determines whether the current cardiopulmonary data is associated with abnormal cardiopulmonary sounds using the Nearest-Neighbor Classification (NNC) algorithm by referencing the normal and abnormal reference data stored in the database 10. In this embodiment, the k-Nearest Neighbors (KNN) Classification is used, with k=3, and the processing device 4 compares the waveform and frequency of the current cardiopulmonary data to those of the normal and abnormal reference data and retrieves, for example, three reference data that are nearest to the current cardiopulmonary data, to determine whether the current cardiopulmonary data is associated with the normal or the abnormal cardiopulmonary sounds according to which one of the normal and abnormal cardiopulmonary sounds being the majority among the three reference data. Note that the number of retrieved reference data is not limited to three and other machine learning algorithms may be employed in other embodiments of this disclosure.

The processing device 4 transmits in real-time a first alert message indicating cardiopulmonary abnormality to the mobile device 2 upon determining that the current cardiopulmonary data is associated with abnormal cardiopulmonary sounds. In one embodiment, the processing device 4 further transmits the current cardiopulmonary data to a medical institute 3 with which the processing device 4 is in communication. In one embodiment, the processing device 4 is capable of obtaining a current position of the mobile device 2 and is further communicatively associated with another mobile device 5, and transmits to the another mobile device 5 a second alert message indicating cardiopulmonary abnormality of the user 20 and containing at least one of the current position of the mobile device 2, a predetermined position of an emergency medical team (not shown) and a predetermined position of an automated external defibrillator (AED). The position of the mobile device 2 may also be transmitted to the medical institute 3 in one embodiment.

In this way, the user 20 of the mobile device 2 can be made aware of cardiopulmonary abnormality in real-time without going to the medical institute 3. Furthermore, a doctor at the medical institute 3 can make his/her own diagnosis based on the current cardiopulmonary data according to his/her clinical experience and make further determinations, such as whether to dispatch ambulance for the user 20. Additionally, a user 50 of the another mobile device 5, who may be the emergency contact (e.g., a friend or relative) of the user 20, may be made aware of the detected abnormality and the current position of the user 20, as well as location of the emergency medical team and/or the AED and can approach the emergency medical team to seek medical assistance for the user 20, or may locate and bring the user 20 to the AED for immediate treatment.

It should be noted that the processing device 4 may be implemented by a server located within the medical institute 3 and the determination of whether the current cardiopulmonary sounds captured from the user 20 is associated with abnormal cardiopulmonary sounds can be made in the medical institute 3 as required. It should be further noted that the communications between the processing device 4 and the mobile device 2, between the processing device 4 and the another mobile device 5, between the processing device 4 and the medical institute 3 and between the processing device 4 and the database 10 may be established differently, as would be appreciated by those skilled in the art.

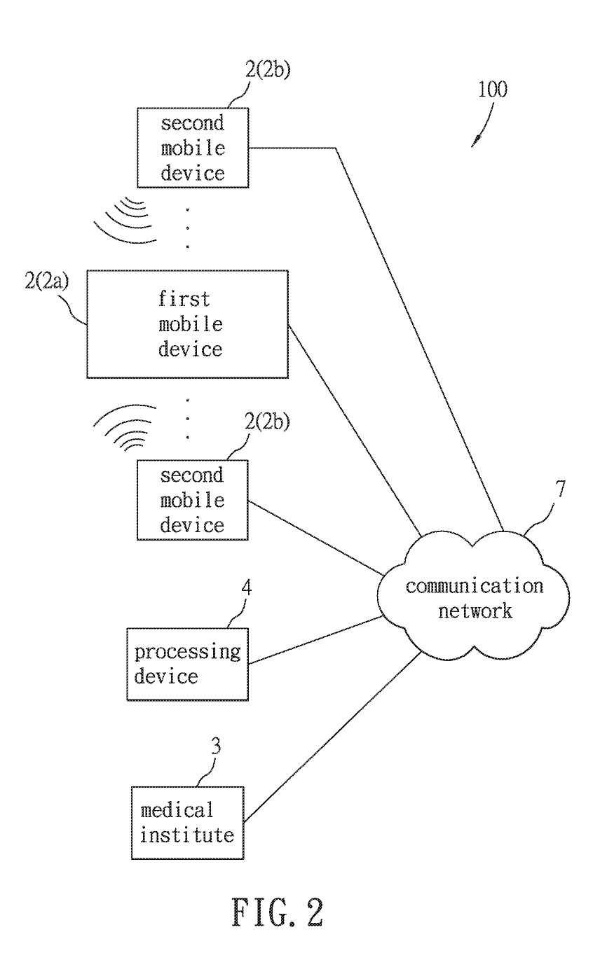

In another embodiment, referring to FIG. 2, the system 100 includes a plurality of mobile devices 2 in communication with each other under a predetermined communication protocol, such as, but not limited to, Bluetooth and near-field communication or over a wireless local area network, and in communication with the processing device 4 over a communication network 7. The mobile devices 2 are capable of acquiring respective current positions of each other, and are each capable of acquiring current cardiopulmonary data associated with current cardiopulmonary sounds from a respective user. The processing device 4 is capable of making a determination as to whether the current cardiopulmonary data received from each of the mobile devices 2 is associated with abnormal cardiopulmonary sounds in a manner discussed above.

Note that in the following description, one of the mobile devices 2, the current cardiopulmonary data received from which is determined to be associated with abnormal cardiopulmonary sounds is referred to as the first mobile device 2a and the other mobile devices 2 are referred to as the second mobile devices 2b. In this embodiment, when it is determined by the processing device 4 that the current cardiopulmonary data is associated with the abnormal cardiopulmonary sounds, the processing device 4 further transmits in real-time an alert message indicating cardiopulmonary abnormality to at least one of the second mobile devices 2b to enable the at least one of the second mobile devices 2b to output the alert message. Aside from indicating that the user of the first mobile device 2a may be suffering from cardiopulmonary abnormality, the alert message may contain at least one of the predetermined position of the emergency medical team and the predetermined position of the automated external defibrillator (AED). In this way, even if the user of the first mobile device 2a is in a coma, a user of at least one second mobile devices 2b can be made aware of cardiopulmonary abnormality of the user of the first mobile device 2a in real-time and to take further appropriate actions, for example, to call 911 or locate the positions of the user of the first mobile device 2a and nearest AED and treat the user with the AED. Similar to the above disclosure, a medical institute 3 may also be informed of the detection of cardiopulmonary abnormality and be provided with the current cardiopulmonary data of the user of the first mobile device 2a.

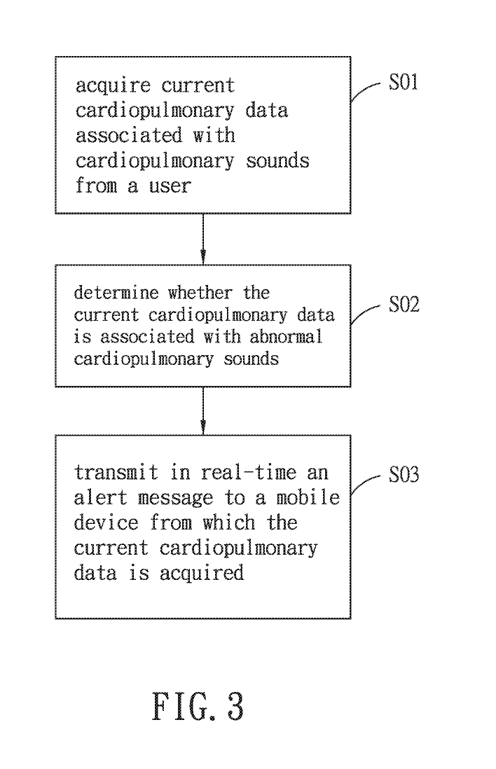

FIG. 3 is a flow chart of a method for detecting cardiopulmonary abnormality according to an embodiment of this disclosure and the method is to be implemented by a processing device 4 that is located remotely of and communicatively associated with a mobile device 2 (see FIG. 1).

In step S01, the processing device 4 acquires the current cardiopulmonary data associated with current cardiopulmonary sounds from a user 20 as acquired by the mobile device 2. In step S02, the processing device 4 determines whether the current cardiopulmonary data is associated with abnormal cardiopulmonary sounds. In step S03, the processing device 4 transmits in real-time an alert message indicating cardiopulmonary abnormality to the mobile device 2 when it is determined in step S02 that the current cardiopulmonary data is associated with abnormal cardiopulmonary sounds. Variations to the method may be implemented based on the above disclosure.

To sum up, the system 100 and the method of this disclosure are capable of detecting and reporting in real-time cardiopulmonary abnormality of the user of the mobile device 2, 2a. Search and rescue time for the user of the mobile device 2, 2a may be reduced for the current position of the mobile device 2 being made available to the user of another mobile devices 5, 2b (either acquired from the alert message or acquired via communication with the mobile device 2a under the communication protocol). The positions of emergency medical team and the AED may also be provided to those who can help. Further, doctors at the medical institute 3 can remotely diagnose illness of the user of the mobile device 2, 2a and make further determinations based on the current cardiopulmonary data received from the processing device 4. As a result, survival rate of the user suffering from diseases relating to myocardial infarction, mitral valve prolapsed, asthma, etc. can be increased.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A system for detecting cardiopulmonary abnormality, comprising:
a mobile device including a cardiopulmonary data acquiring module that is for acquiring current cardiopulmonary data associated with current cardiopulmonary sounds from a user; and
a processing device located remotely of and communicatively associated with said mobile device, receiving the current cardiopulmonary data from said mobile device, determining whether the current cardiopulmonary data is associated with abnormal cardiopulmonary sounds, and transmitting in real-time a first alert message indicating cardiopulmonary abnormality to said mobile device upon determining that the current cardiopulmonary data is associated with abnormal cardiopulmonary sounds.

2. The system for detecting cardiopulmonary abnormality as claimed in claim 1, wherein said processing device determines whether the current cardiopulmonary data is associated with abnormal cardiopulmonary sounds through machine learning with reference to a plurality of normal reference data associated with different normal cardiopulmonary sounds and a plurality of abnormal reference data associated with different abnormal cardiopulmonary sounds.

3. The system for detecting cardiopulmonary abnormality as claimed in claim 2, further comprising a database storing the normal reference data and the abnormal reference data, and accessible by said processing device, wherein said processing device determining whether the current cardiopulmonary data is associated with abnormal cardiopulmonary sounds using the Nearest-Neighbor Classification (NNC) algorithm by referencing the normal and abnormal reference data stored in said database.

4. The system for detecting cardiopulmonary abnormality as claimed in claim 1, wherein said cardiopulmonary data acquiring module of said mobile device includes a cardiopulmonary sound capturing unit for capturing the current cardiopulmonary sounds from the user, and a processing unit for converting the current cardiopulmonary sounds captured by said cardiopulmonary sound capturing unit into the current cardiopulmonary data, the current cardiopulmonary data being represented by waveform and frequency.

5. The system for detecting cardiopulmonary abnormality as claimed in claim 1, further comprising another mobile device communicatively associated with said mobile device, wherein said processing device transmits a second alert message to said another mobile device when it is determined by said processing device that the current cardiopulmonary data is associated with abnormal cardiopulmonary sounds, to enable said another mobile device to output the second alert message.

6. The system for detecting cardiopulmonary abnormality as claimed in claim 5, wherein said processing device is capable of obtaining a current position of said mobile device, and the second alert message includes at least one of the current position of said mobile device, a predetermined position of an emergency medical team and a predetermined position of an automated external defibrillator (AED).

7. The system for detecting cardiopulmonary abnormality as claimed in claim 1, wherein said processing device transmits the current cardiopulmonary data to a medical institute when it is determined thereby that the current cardiopulmonary data is associated with abnormal cardiopulmonary sounds.

8. A system for reporting cardiopulmonary abnormality, comprising:
a first mobile device including a cardiopulmonary data acquiring module that is for acquiring current cardiopulmonary data associated with current cardiopulmonary sounds from a user;
a second mobile device communicatively associated with said first mobile device; and
a processing device located remotely of and communicatively associated with said first and second mobile devices, receiving the current cardiopulmonary data from said first mobile device, and determining whether the current cardiopulmonary data is associated with abnormal cardiopulmonary sounds;
wherein said processing device transmits in real-time an alert message indicating cardiopulmonary abnormality to said second mobile device upon determining that the current cardiopulmonary data from said first mobile device is associated with abnormal cardiopulmonary sounds.

9. The system for reporting cardiopulmonary abnormality as claimed in claim 8, wherein said processing device is capable of acquiring a current position of said first mobile device, and the alert message contains the current position of said first mobile device.

10. The system for reporting cardiopulmonary abnormality as claimed in claim 9, wherein the alert message further contains one of a predetermined position of an emergency medical team and a predetermined position of an automated external defibrillator (AED)

11. The system for reporting cardiopulmonary abnormality as claimed in claim 8, wherein said second mobile device is capable of acquiring a current position of said first mobile device.

12. The system for reporting cardiopulmonary abnormality as claimed in claim 8, wherein said processing device further transmits in real-time the current cardiopulmonary data from said first mobile device to a medical institute upon determining that the current cardiopulmonary data from said first mobile device is associated with abnormal cardiopulmonary sounds.

13. The system for reporting cardiopulmonary abnormality as claimed in claim 8, wherein said processing device determines whether the current cardiopulmonary data is associated with abnormal cardiopulmonary sounds through machine learning with reference to a plurality of normal reference data associated with different normal cardiopulmonary sounds and a plurality of abnormal reference data associated with different abnormal cardiopulmonary sounds.

14. The system for reporting cardiopulmonary abnormality as claimed in claim 13, further comprising a database storing the normal reference data and the abnormal reference data, and accessible by said processing device, wherein said processing device determining whether the current cardiopulmonary data is associated with abnormal cardiopulmonary sounds using the Nearest-Neighbor Classification (NNC) algorithm by referencing the normal and abnormal reference data stored in said database.

15. A method for detecting cardiopulmonary abnormality, to be implemented by a processing device that is located remotely of and communicatively associated with a mobile device, the method comprising the steps of:
d) acquiring, by the processing device, current cardiopulmonary data associated with cardiopulmonary sounds from a user as acquired by the mobile device;
e) determining, by the processing device, whether the current cardiopulmonary data is associated with abnormal cardiopulmonary sounds; and
f) transmitting in real-time, by the processing device, an alert message indicating cardiopulmonary abnormality to the mobile device when it is determined in step b) that the current cardiopulmonary data is associated with abnormal cardiopulmonary sounds.
